SEQUENCE LISTING (10 sequences)

SEQ ID NO: 1 - protein, 1709 aa, Homo sapiens
MKKIFSKKGESPLGSFARRQRSSAGGGGEPGEGAYSQPGYHVRDRDLGKIHKAASAGNVAKVQQILLLRKNGLNDRDKMNRTALHLACANGHPEVVTLLVDRKCQLNVCDNENRTALMKAVQCQEEKCATILLEHGADPNLADVHGNTALHYAVYNEDISVATKLLLYDANIEAKNKDDLTPLLLAVSGKKQQMVEFLIKKKANVNAVDKLESSHQLISEYKEERIPKHSSQNSNSVDESSEDSLSRLSGKPGVDDSWPTSDDEDLNFDTKNVPKPSLAKLMTASQQSRKNLEATYGTVRTGNRTLFEDRDSDSQDEVVVESLPTTSIKVQCFSHPTYQSPDLLPKPSHKSLANPGLMKEEPTKPGIAKKENGIDIIESAPLEQTNNDNLTYVDEVHKNNRSDMMSALGLGQEEDIESPWDSESISENFPQKYVDPLAGAADGKEKNIGNEQAEDVFYIPSCMSGSRNFKMAKLEDTRNVGMPVAHMESPERYLHLKPTIEMKDSVPNKAGGMKDVQTSKAAEHDLEVASEEEQEREGSENNQPQVEEERKKHRNNEMEVSANIHDGATDDAEDDDDDDGLIQKRKSGETDHQQFPRKENKEYASGPALQMKEVKSTEKEKRTSKESVNSPVFGKASLLTGGLLQVDDDSSLSEIDEDEGRPTKKTSNEKNKVKNQIQSMDDVDDLTQSSETASEDCELPHSSYKNFMLLIEQLGMECKDSVSLLKIQDAALSCERLLELKKNHCELLTVKIKKMEDKVNVLQRELSETKEIKSQLEHQKVEWERELCSLRFSLNQEEEKRRNADTLYEKIREQLRRKEEQYRKEVEVKQQLELSLQTLEMELRTVKSNLNQVVQERNDAQRQLSREQNARMLQDGILTNHLSKQKEIEMAQKKMNSENSHSHEEEKDLSHKNSMLQEEIAMLRLEIDTIKNQNQEKEKKCFEDLKIVKEKNEDLQKTIKQNEETLTQTISQYNGRLSVLTAENAMLNSKLENEKQSKERLEAEVESYHSRLAAAIHDRDQSETSKRELELAFQRARDECSRLQDKMNFDVSNLKDNNEILSQQLFKTESKLNSLEIEFHHTRDALREKTLGLERVQKDLSQTQCQMKEMEQKYQNEQVKVNKYIGKQESVEERLSQLQSENMLLRQQLDDAHNKADNKEKTVINIQDQFHAIVQKLQAESEKQSLLLEERNKELISECNHLKERQYQYENEKAEREVVVRQLQQELADTLKKQSMSEASLEVTSRYRINLEDETQDLKKKLGQIRNQLQEAQDRHTEAVRCAEKMQDHKQKLEKDNAKLKVTVKKQMDKIEELQKNLLNANLSEDEKEQLKKLMELKQSLECNLDQEMKKNVELEREITGFKNLLKMTRKKLNEYENGEFSFHGDLKTSQFEMDIQINKLKHKIDDLTAELETAGSKCLHLDTKNQILQEELLSMKTVQKKCEKLQKNKKKLEQEVINLRSHIERNMVELGQVKQYKQEIEERARQEIAEKLKEVNLFLQAQAASQENLEQFRENNFASMKSQMELRIKDLESELSKIKTSQEDFNKTELEKYKQLYLEELKVRKSLSSKLTKTNERLAEVNTKLLVEKQQSRSLFTTLTTRPVMEPPCVGNLNNSLDLNRKLIPRENLVISTSNPRASNNSMENYLSKMQQELEKNITRELKEAAAELESGSIASPLGSTDESNLNQDLVWKASREYVQVLKKNYMI

SEQ ID NO: 2 - DNA, 6776 bp, homo sapiens
acttgtgtctagcgggatcgcttgcttggtaacccggagggagagattggaaaccgcggagtttcctttgggaggctgcggccagccggggctgacttgttatgttgggctccggaggccgttaagagccgagagagacatgaggtgtctctgaagcccggtcgcctgggccatgaagaagatttttagtaagaagggcgagtcgcccttgggctccttcgcgcggcggcagaggagcagcgcgggaggcgggggcgagccgggggagggcgcctactcgcagcccggctaccacgtccgagaccgagatctcggcaagatccacaaagctgccagcgcgggtaatgtggcgaaagtgcagcagatccttttgctcaggaagaatggcttgaacgatagagacaagatgaacaggacggctctacatttggcctgtgccaatggtcatccagaagtagtaactctcctggtggacagaaaatgccagctcaatgtctgtgacaacgaaaacaggacagctctgatgaaggctgtacaatgccaggaagagaaatgtgcaactattctgctagaacatggtgctgatccaaatcttgcggatgtccatggcaacactgctcttcactatgctgtctataatgaggacatatcagtagcaacaaagctgcttttgtatgatgcaaatattgaagcaaaaaacaaggatgacctcacaccacttttacttgcagtaagtggaaaaaagcagcaaatggtggaatttttaataaagaaaaaagcaaatgtaaatgcagtagataagttggaaagcagtcaccaactaatttcagaatataaagaagaaaggatacctaaacattcttctcaaaatagtaattcagtggatgaaagctctgaagactccttaagcaggctttctggcaaaccgggtgttgatgattcatggcctacctcagatgacgaagacctcaattttgatactaagaatgtcccaaaaccaagcttagcaaagctaatgactgcttctcagcaatccaggaaaaatttagaagcaacatatggcactgtgagaacaggaaatagaactttgtttgaggatagagattccgatagtcaagatgaagttgtggttgaaagccttcctacaacatcaatcaaagtccagtgcttttctcatcctacctatcaatcacctgaccttcttccaaaaccttcccacaagtcgttagcaaaccctggtcttatgaaggaagaaccaacaaagccaggcattgcaaaaaaagaaaatggtattgatattattgaaagtgctccactagagcaaacaaataatgacaatttgacttatgttgatgaagtgcacaaaaataatagaagtgatatgatgtccgcattaggattaggacaagaggaagatatagaatcaccttgggattctgagagtatctctgagaattttccacagaagtatgttgatcctttagctggggctgcagacggaaaagaaaaaaatataggaaatgaacaagcagaagatgtgttttatataccttcttgcatgagtggatcaagaaactttaagatggctaaactagaggatacaagaaatgtaggcatgccagtagcccacatggagtctcctgagagatatcttcacttgaagcctaccattgaaatgaaagattctgttccaaataaagcaggaggaatgaaggatgtacaaacatccaaagcagctgaacatgacttagaagtagcatcagaagaagagcaagaaagggaagggagtgaaaataaccagccacaggttgaagaagaaaggaaaaaacacagaaataatgaaatggaagtatcagcaaacatacatgatggtgctactgatgatgctgaagatgatgatgatgatgatggattaattcaaaaaagaaagagtggagaaactgatcatcagcaatttcccaggaaggaaaataaagagtatgctagtggtcctgccttgcaaatgaaggaagtaaagagcactgaaaaagaaaaacggacctcgaaagaatctgtgaattcaccagtgtttgggaaggccagtttactaactggtggcctgctacaagtggatgatgacagcagtttaagtgaaatagatgaggatgaaggaaggcctactaagaaaacatctaatgaaaagaacaaggtcaaaaaccaaatacagtctatggatgatgttgatgacttaactcagtcatctgaaacagcctcagaggattgtgagctaccccactctagttacaagaattttatgttgctcattgaacaacttggaatggagtgtaaagattctgttagcctattgaaaatccaggatgcagctctttcatgtgaaagattattagaacttaaaaaaaatcactgtgaactacttacagtaaaaattaaaaaaatggaagacaaggttaatgtactacaaagggagctatctgaaacaaaagaaataaaatcacagttagagcatcaaaaagttgaatgggaacgagaactgtgctctttgagatttagcttaaaccaagaagaagagaagagaagaaatgctgatacgttgtatgaaaaaattagggaacagttaagaagaaaagaagagcaatataggaaagaagttgaagtgaaacaacagcttgaactgagtctccaaacactggagatggaattgaggactgtaaaaagtaatttgaatcaggtcgttcaagagcgaaatgacgctcagaggcaactttctcgagaacagaatgccagaatgttacaagatggaattctgaccaatcacctttccaaacaaaaggagattgaaatggctcaaaagaaaatgaattctgagaattctcatagtcatgaagaagaaaaagacctatcgcataaaaatagcatgttgcaggaagaaattgctatgctaagactagaaatagacacaataaaaaatcaaaaccaggaaaaagaaaagaaatgttttgaggaccttaaaattgtaaaagaaaagaatgaagaccttcagaagactataaaacagaatgaggaaacattaacacaaacaatatcccagtataatggacggcttagtgttctgacagctgagaatgcaatgctaaattctaaactggagaatgaaaagcaaagcaaggaaagactggaagcagaagttgaatcataccattctagattggctgctgctatacatgatcgtgatcaaagtgagacatcaaaaagagaactagaacttgctttccagagagcaagagatgaatgttctcgtttacaggacaaaatgaattttgatgtgtctaacctaaaagataacaatgagattctttctcaacaactatttaaaactgaaagtaaactcaatagcctagaaattgagttccatcacacgagagatgccctcagagaaaagactttgggtttagaacgggtacaaaaggacctaagccaaacacagtgtcaaatgaaggaaatggaacaaaagtatcaaaatgaacaagttaaagtgaataaatacattggaaagcaggagtctgtagaggagagattgtctcaactacaaagtgagaatatgttgcttcgacaacaactggatgatgcccacaacaaggctgacaataaagagaagacagtgattaatatccaagaccagtttcatgctattgtgcaaaaacttcaagctgagagtgaaaagcaaagtcttctgctagaagaaagaaataaggagttaatcagtgaatgtaatcacttaaaagaaagacagtatcaatatgaaaatgaaaaggcagaaagagaagttgttgtgagacaacttcaacaagaactagctgataccctaaaaaaacaatctatgtcagaggcttcactggaggttacgtcacgttatcgtattaatttagaagatgagacacaggatttaaagaagaaattaggtcaaatcagaaatcaattgcaagaagcacaggatcgacatacagaagctgtcagatgtgctgagaagatgcaagatcacaagcaaaagcttgaaaaagataatgccaagttaaaagttacagtcaaaaagcaaatggacaaaattgaggagcttcagaaaaacctgttaaatgcaaatttgtctgaagatgaaaaggaacaattaaagaaacttatggaattaaaacagtcactggaatgtaatttggatcaagaaatgaagaaaaatgttgaattagaaagagagataactggatttaagaacctcttaaaaatgacaagaaagaagttaaatgaatatgaaaatggagaatttagtttccatggagatttaaaaactagtcaatttgaaatggatattcagattaataagctaaaacataagattgatgatcttacagcagaactggagactgcaggttcaaaatgtctacatctggatacaaagaaccaaattcttcaagaggagttgttatctatgaaaacagtacaaaagaaatgtgaaaaactacagaagaataaaaagaagttggaacaagaagtgatcaacctgagaagtcatatagaaaggaatatggtagaacttggtcaagtcaaacagtataaacaggagattgaagaaagagcaagacaggaaatagcagaaaaattaaaagaagtcaatctatttttacaggcacaagcagcatctcaagaaaacttagagcagtttagagagaataattttgcttcaatgaaaagtcagatggaactcagaattaaagatctggaatctgaactctccaaaataaaaacttctcaagaagactttaataaaaccgaactggaaaaatataagcaactctatctagaagaattaaaagttagaaaatctttgtcaagtaaactaaccaaaactaatgagaggctagcagaggtcaacaccaaacttcttgtggaaaaacagcagagcagatctttgttcaccactctcactaccaggccagtcatggagccaccttgtgtgggaaatcttaataatagtttagatctcaacagaaaacttattccaagagaaaacttagtgatctctacctcaaatccacgggcttcaaataatagcatggagaactacttgagcaagatgcagcaggagttggaaaaaaatataactagagaactcaaagaagctgctgctgaattggaatctggatcaatagcttcccctctagggtctactgatgagtcaaatctaaatcaagatctagtttggaaagcatcaagagaatatgtacaggttttaaagaaaaattatatgatctgaaagataagattttattactgggctgtttatgtgacattttgtttctcattaaatattaatatatgacatgtaaaaatattaaaggaacgtatttttgtatcatatatgtcagatcaaaatttatatagtattttaaacaatgttacttggcttctttaacttttaagtggattttgccaatgaaaaccaggaatattgagttttacatactcaaactgcccaaatgtgagctgtttaaacagccaaaccactaagttgtcattaatactttagtgcagttaattgatggcttatttgtattttgtaatttttatcactatatgtagtggtagatgtctaattggacttagacatcattttgttgtgcaggaactgtggtcattgttcatgtttggatgtattacaattgttacagtgccatcaaatttacataattttaattttaagcactgatttatatagctgttctcagggagaaacaagatttgcctgattcaccttttctgtttgtttatttatttttaattaaccttttggaaatagccttcagtttagagaaaatttcgaagttttagtacaaagactttctcctccgaacaatttgagagtaaattgctgacattatgtcatcacccctgattactttagtgggcattcctacaagtcaggacattctacataactacagtgcaaaccttgcaatcagaaagtcaacattaatacattctagtttgttacttctctgtaatgtcctttatagcaaaaggatcccattctaatccatggcttgtatttagttgttgtatctgttagatctttcagtctggaacagttcctcagtctttccttgaccttcatgaccttgaatattttgaagagtaaagtctggtaatataagaatgtccctcaattgggtttcatggtgattagatttagattatatgattttttggcaggagtatcacagaagtgacccagtgatcttctcattgcatcttacctgtgaccctacagttttgatttgccctgttactggtaatgttagcattgatcactaacagtggtatctgctgaatttctcctctgtaatgttattttttcaccttgtggttaattagtattttacagggaggtaatttgagactatgtacatatctctactccccatcgaaccctcaatttattcatttgcttgtatcaaataaaccatggattcatggtttattttattcagtgagttataattcattaatattgttattaaaaatttgtttttcttttaagaaatgttatattttgacaacttcagacttacagaaagattataagaggtagtacaaataatttttggatattattccccaaatgttaacattttactgcatttactttatcctttctcccccttctcctcctgtctttctagatgaatatgaatataggtacttaatacagattttttttctaaactgtt

SEQ ID NO: 3 - protein, 1976 aa, homo sapiens
MAQRTGLEDPERYLFVDRAVIYNPATQADWTAKKLVWIPSERHGFEAASIKEERGDEVMVELAENGKKAMVNKDDIQKMNPPKFSKVEDMAELTCLNEASVLHNLKDRYYSGLIYTYSGLFCVVINPYKNLPIYSENIIEMYRGKKRHEMPPHIYAISESAYRCMLQDREDQSILCTGESGAGKTENTKKVIQYLAHVASSHKGRKDHNIPGELERQLLQANPILESFGNAKTVKNDNSSRFGKFIRINFDVTGYIVGANIETYLLEKSRAVRQAKDERTFHIFYQLLSGAGEHLKSDLLLEGFNNYRFLSNGYIPIPGQQDKDNFQETMEAMHIMGFSHEEILSMLKVVSSVLQFGNISFKKERNTDQASMPENTVAQKLCHLLGMNVMEFTRAILTPRIKVGRDYVQKAQTKEQADFAVEALAKATYERLFRWLVHRINKALDRTKRQGASFIGILDIAGFEIFELNSFEQLCINYTNEKLQQLFNHTMFILEQEEYQREGIEWNFIDFGLDLQPCIDLIERPANPPGVLALLDEECWFPKATDKTFVEKLVQEQGSHSKFQKPRQLKDKADFCIIHYAGKVDYKADEWLMKNMDPLNDNVATLLHQSSDRFVAELWKDVDRIVGLDQVTGMTETAFGSAYKTKKGMFRTVGQLYKESLTKLMATLRNTNPNFVRCIIPNHEKRAGKLDPHLVLDQLRCNGVLEGIRICRQGFPNRIVFQEFRQRYEILTPNAIPKGFMDGKQACERMIRALELDPNLYRIGQSKIFFRAGVLAHLEEERDLKITDIIIFFQAVCRGYLARKAFAKKQQQLSALKVLQRNCAAYLKLRHWQWWRVFTKVKPLLQVTRQEEELQAKDEELLKVKEKQTKVEGELEEMERKHQQLLEEKNILAEQLQAETELFAEAEEMRARLAAKKQELEEILHDLESRVEEEEERNQILQNEKKKMQAHIQDLEEQLDEEEGARQKLQLEKVTAEAKIKKMEEEILLLEDQNSKFIKEKKLMEDRIAECSSQLAEEEEKAKNLAKIRNKQEVMISDLEERLKKEEKTRQELEKAKRKLDGETTDLQDQIAELQAQIDELKLQLAKKEEELQGALARGDDETLHKNNALKVVRELQAQIAELQEDFESEKASRNKAEKQKRDLSEELEALKTELEDTLDTTAAQQELRTKREQEVAELKKALEEETKNHEAQIQDMRQRHATALEELSEQLEQAKRFKANLEKNKQGLETDNKELACEVKVLQQVKAESEHKRKKLDAQVQELHAKVSEGDRLRVELAEKASKLQNELDNVSTLLEEAEKKGIKFAKDAASLESQLQDTQELLQEETRQKLNLSSRIRQLEEEKNSLQEQQEEEEEARKNLEKQVLALQSQLADTKKKVDDDLGTIESLEEAKKKLLKDAEALSQRLEEKALAYDKLEKTKNRLQQELDDLTVDLDHQRQVASNLEKKQKKFDQLLAEEKSISARYAEERDRAEAEAREKETKALSLARALEEALEAKEEFERQNKQLRADMEDLMSSKDDVGKNVHELEKSKRALEQQVEEMRTQLEELEDELQATEDAKLRLEVNMQAMKAQFERDLQTRDEQNEEKKRLLIKQVRELEAELEDERKQRALAVASKKKMEIDLKDLEAQIEAANKARDEVIKQLRKLQAQMKDYQRELEEARASRDEIFAQSKESEKKLKSLEAEILQLQEELASSERARRHAEQERDELADEITNSASGKSALLDEKRRLEARIAQLEEELEEEQSNMELLNDRFRKTTLQVDTLNAELAAERSAAQKSDNARQQLERQNKELKAKLQELEGAVKSKFKATISALEAKIGQLEEQLEQEAKERAAANKLVRRTEKKLKEIFMQVEDERRHADQYKEQMEKANARMKQLKRQLEEAEEEATRANASRRKLQRELDDATEANEGLSREVSTLKNRLRRGGPISFSSSRSGRRQLHLEGASLELSDDDTESKTSDVNETQPPQSE

SEQ ID NO: 4 - DNA, 7685 bp, homo sapiens
agcagtgctaaaggagcccggcggaggcagcggtgggtttgggactgaggcgctggatctgtggtcgcggctggggacgtgcgcccgcgccaccatcttcggctgaagaggcaattgcttttggatcgttccatttacaatggcgcagagaactggactcgaggatccagagaggtatctctttgtggacagggctgtcatctacaaccctgccactcaagctgattggacagctaaaaagctagtgtggattccatcagaacgccatggttttgaggcagctagtatcaaagaagaacggggagatgaagttatggtggagttggcagagaatggaaagaaagcaatggtcaacaaagatgatattcagaagatgaacccacctaagttttccaaggtggaggatatggcagaattgacatgcttgaatgaagcttccgttttacataatctgaaggatcgctactattcaggactaatctatacttattctggactcttctgtgtagttataaacccttacaagaatcttccaatttactctgagaatattattgaaatgtacagagggaagaagcgtcatgagatgcctccacacatctatgctatatctgaatctgcttacagatgcatgcttcaagatcgtgaggaccagtcaattctttgcacgggtgagtcaggtgctgggaagacagaaaatacaaagaaagttattcagtaccttgcccatgttgcttcttcacataaaggaagaaaggaccataatattcctggggaacttgaacggcagcttttgcaagcaaatccaattctggaatcatttggaaatgcgaagactgtgaaaaatgataactcatctcgttttggcaaatttattcggatcaactttgatgtaactggctatatcgttggggccaacattgaaacataccttctggaaaagtctcgtgctgttcgtcaagcaaaagatgaacgtacttttcatatcttttaccagttgttatctggagcaggagaacacctaaagtctgatttgcttcttgaaggatttaataactacaggtttctctccaatggctatattcctattccgggacagcaagacaaagataatttccaggagaccatggaagcaatgcacataatgggcttctcccatgaagagattctgtcaatgcttaaagtagtatcttcagtgctacagtttggaaatatttctttcaaaaaggagagaaatactgatcaagcttccatgccagaaaatacagttgcgcagaagctctgccatcttcttgggatgaatgtgatggagtttactcgggccatcctgactccccggatcaaggtcggccgagactatgtgcaaaaagcccagaccaaagaacaggcagattttgcagtagaagcattggcaaaagctacctatgagcggctctttcgctggctcgttcatcgcatcaataaagctctggataggaccaaacgtcagggagcatctttcattggaatcctggatattgctggatttgaaatttttgagctgaactcctttgaacaactttgcatcaactacaccaatgagaagctgcagcagctgttcaaccacaccatgtttatcctagaacaagaggaataccagcgcgaaggcatcgagtggaacttcatcgatttcgggctggatctgcagccatgcatcgacctaatagagagacctgcgaaccctcctggtgtactggcccttttggatgaagaatgctggttccctaaagccacagataaaacctttgttgaaaaactggttcaagagcaaggttcccactccaagtttcagaaacctcgacaattaaaagacaaagctgatttttgcattatacattatgcagggaaggtggactataaggcagatgagtggctgatgaagaatatggaccccctgaatgacaacgtggccacccttttgcaccagtcatcagacagatttgtggcagagctttggaaagatgtggaccgtatcgtgggtctggatcaagtcactggtatgactgagacagcttttggctccgcatataaaaccaagaagggcatgtttcgtaccgttgggcaactctacaaagaatctctcaccaagctgatggcaactctccgaaacaccaaccctaactttgttcgttgtatcattccaaatcacgagaagagggctggaaaattggatccacacctagtcctagatcagcttcgctgtaatggtgtcctggaagggatccgaatctgtcgccagggcttccctaaccgaatagttttccaggaattcagacagagatatgagatcctaactccaaatgctattcctaaaggttttatggatggtaaacaggcctgtgaacgaatgatccgggctttagaattggacccaaacttgtacagaattggacagagcaagatatttttcagagctggagttctggcacacttagaggaagaaagagatttaaaaatcaccgatatcattatcttcttccaggccgtttgcagaggttacctggccagaaaggcctttgccaagaagcagcagcaactaagtgccttaaaggtcttgcagcggaactgtgccgcgtacctgaaattacggcactggcagtggtggcgagtcttcacaaaggtgaagccgcttctacaagtgactcgccaggaggaagaacttcaggccaaagatgaagagctgttgaaggtgaaggagaagcagacgaaggtggaaggagagctggaggagatggagcggaagcaccagcagcttttagaagagaagaatatccttgcagaacaactacaagcagagactgagctctttgctgaagcagaagagatgagggcaagacttgctgctaaaaagcaggaattagaagagattctacatgacttggagtctagggttgaagaagaagaagaaagaaaccaaatcctccaaaatgaaaagaaaaaaatgcaagcacatattcaggacctggaagaacagctagacgaggaggaaggggctcggcaaaagctgcagctggaaaaggtgacagcagaggccaagatcaagaagatggaagaggagattctgcttctcgaggaccaaaattccaagttcatcaaagaaaagaaactcatggaagatcgcattgctgagtgttcctctcagctggctgaagaggaagaaaaggcgaaaaacttggccaaaatcaggaataagcaagaagtgatgatctcagatttagaagaacgcttaaagaaggaagaaaagactcgtcaggaactggaaaaggccaaaagaaaactcgacggggagacgaccgacctgcaggaccagatcgcagagctgcaggcgcagattgatgagctcaagctgcagctggccaagaaggaggaggagctgcagggcgcactggccagaggtgatgatgaaacactccataagaacaatgcccttaaagttgtgcgagagctacaagcccaaattgctgaacttcaggaagactttgaatccgagaaggcttcacggaacaaggccgaaaagcagaaaagggacttgagtgaggaactggaagctctgaaaacagagctggaggacacgctggacaccacggcagcccagcaggaactacgtacaaaacgtgaacaagaagtggcagagctgaagaaagctcttgaggaggaaactaagaaccatgaagctcaaatccaggacatgagacaaagacacgcaacagccctggaggagctctcagagcagctggaacaggccaagcggttcaaagcaaatctagagaagaacaagcagggcctggagacagataacaaggagctggcgtgtgaggtgaaggtcctgcagcaggtcaaggctgagtctgagcacaagaggaagaagctcgacgcgcaggtccaggagctccatgccaaggtctctgaaggcgacaggctcagggtggagctggcggagaaagcaagtaagctgcagaatgagctagataatgtctccacccttctggaagaagcagagaagaagggtattaaatttgctaaggatgcagctagtcttgagtctcaactacaggatacacaggagcttcttcaggaggagacacgccagaaactaaacctgagcagtcggatccggcagctggaagaggagaagaacagtcttcaggagcagcaggaggaggaggaggaggccaggaagaacctggagaagcaagtgctggccctgcagtcccagttggctgataccaagaagaaagtagatgacgacctgggaacaattgaaagtctggaagaagccaagaagaagcttctgaaggacgcggaggccctgagccagcgcctggaggagaaggcactggcgtatgacaaactggagaagaccaagaaccgcctgcagcaggagctggacgacctcacggtggacctggaccaccagcgccaggtcgcctccaacttggagaagaagcagaagaagtttgaccagctgttagcagaagagaagagcatctctgctcgctatgccgaagagcgggaccgggccgaagccgaggccagagagaaagaaaccaaagccctgtcactggcccgggccctcgaggaagccctggaggccaaggaggagtttgagaggcagaacaagcagctccgagcagacatggaagacctcatgagctccaaagatgatgtgggaaaaaacgttcacgaacttgaaaaatccaaacgggccctagagcagcaggtggaggaaatgaggacccagctggaggagctggaagacgaactccaggccacggaagatgccaagcttcgtctggaggtcaacatgcaggccatgaaggcgcagttcgagagagacctgcaaaccagggatgagcagaatgaagagaagaagcggctgctgatcaaacaggtgcgggagctcgaggcggagctggaggatgagaggaaacagcgggcgcttgctgtagcttcaaagaaaaagatggagatagacctgaaggacctcgaagcccaaatcgaggctgcgaacaaagctcgggatgaggtgattaagcagctccgcaagctccaggctcagatgaaggattaccaacgtgaattagaagaagctcgtgcatccagagatgagatttttgctcaatccaaagagagtgaaaagaaattgaagagtctggaagcagaaatccttcaattgcaggaggaacttgcctcatctgagcgagcccgccgacacgccgagcaggagagagatgagctggcggacgagatcaccaacagcgcctctggcaagtccgcgctgctggatgagaagcggcgtctggaagctcggatcgcacagctggaggaggagctggaagaggagcagagcaacatggagctgctcaacgaccgcttccgcaagaccactctacaggtggacacactgaacgccgagctagcagccgagcgcagcgccgcccagaagagtgacaatgcacgccagcaactggagcggcagaacaaggagctgaaggccaagctgcaggaactcgagggtgctgtcaagtctaagttcaaggccaccatctcagccctggaggccaagattgggcagctggaggagcagcttgagcaggaagccaaggaacgagcagccgccaacaaattagtccgtcgcactgagaagaagctgaaagaaatcttcatgcaggttgaggatgagcgtcgacacgcggaccagtataaagagcagatggagaaggccaacgctcggatgaagcagcttaaacgccagctggaggaagcagaagaagaagcgacgcgtgccaacgcatctcggcgtaaactccagcgggaactggatgatgccaccgaggccaacgagggcctgagccgcgaggtcagcaccctgaagaaccggctgaggcggggtggccccatcagcttctcttccagccgatctggccggcgccagctgcaccttgaaggagcttccctggagctctccgacgatgacacagaaagtaagaccagtgatgtcaacgagacgcagccaccccagtcagagtaaagttgcaggaagccagaggaggcaatacagtgggacagttaggaatgcacccggggcctcctgcagatttcggaaattggcaagctacgggattccttcctgaaagatcaactgtgtcttaaggctctccagcctatgcatactgtatcctgcttcagacttaggtacaattgctcccctttttatatatagacacacacaggacacatatattaaacagattgtttcatcattgcatctattttccatatagtcatcaagagaccattttataaaacatggtaagaccctttttaaaacaaactccaggcccttggttgcgggtcgctgggttattggggcagcgccgtggtcgtcactcagtcgctctgcatgctctctgtcatacagacaggtaacctagttctgtgttcacgtggcccccgactcctcagccacatcaagtctcctagaccactgtggactctaaactgcacttgtctctctcatttccttcaaataatgatcaatgctatttcagtgagcaaactgtgaaaggggctttggaaagagtaggaggggtgggctggatcggaagcaacacccatttggggttaccatgtccatcccccaaggggggccctgcccctcgagtcgatggtgtcccgcatctactcatgtgaactggccttggcgagggctggtctgtgcatagaagggatagtggccacactgcagctgaggccccaggtggcagccatggatcatgtagacttccagatggtctcccgaaccgcctggctctgccggcgccctcctcacgtcaggagcaagcagccgtggacccctaagccgagctggtggaaggcccctccccgtcgccagccgggccctcatgctgaccttgcaaattcagccgctgctttgagcccaaaatgggaatattggttttgtgtccgaggcttgttccaagtttgtcaatgaggtttatggagcctccagaacagatgccatcttcctgaatgttgacatgccagtgggtgtgactccttcatttttccttctcccttccctttggacagtgttacagtgaacacttagcatcctgtttttggttggtagttaagcaaactgacattacggaaagtgccttagacactacagtactaagacaatgttgaatatatcattcgcctctataacaatttaatgtattcagttttgactgtgcttcatatcatgtacctctctagtcaaagtggtattacagacattcagtgacaatgaatcagtgttaattctaaatccttgatcctctgcaatgtgcttgaaaacacaaaccttttgggttaaaagctttaacatctattaggaagaatttgtcctgtgggtttggaatcttggattttccccctttatgaactgtactggctgttgaccaccagacacctgaccgcaaatatcttttcttgtattcccatatttctagacaatgatttttgtaagacaataaatttattcattatagatatttgcgcctgctctgtttacttgaagaaaaaagcacccgtggagaataaagagacctcaataaacaagaataatcatgtgaacgtggaaaaaaaaaaaaaaaaaa

SEQ ID NO: 5 - DNA, 23 bp, Artificial Sequence
  primers
ctatgtcagaggcttcactggag

SEQ ID NO: 6 - DNA, 23 bp, Artificial Sequence
  primers
ctcagcacatctgacagcttctg

SEQ ID NO: 7 - DNA, 22 bp, Artificial Sequence
  primers
gctgatggcaactctccgaaac

SEQ ID NO: 8 - DNA, 22 bp, Artificial Sequence
  primers
cttccaggacaccattacagcg

SEQ ID NO: 9 - protein, 1710 aa, Homo sapiens
MKKIFSKKGESPLGSFARRQRSSAGGGGEPGEGAYSQPGYHVRDRDLGKIHKAASAGNVAKVQQILLLRKNGLNDRDKMNRTALHLACANGHPEVVTLLVDRKCQLNVCDNENRTALMKAVQCQEEKCATILLEHGADPNLADVHGNTALHYAVYNEDISVATKLLLYDANIEAKNKDDLTPLLLAVSGKKQQMVEFLIKKKANVNAVDKLESSHQLISEYKEERIPKHSSQNSNSVDESSEDSLSRLSGKPGVDDSWPTSDDEDLNFDTKNVPKPSLAKLMTASQQSRKNLEATYGTVRTGNRTLFEDRDSDSQDEVVVESLPTTSIKVQCFSHPTYQSPDLLPKPSHKSLANPGLMKEEPTKPGIAKKENGIDIIESAPLEQTNNDNLTYVDEVHKNNRSDMMSALGLGQEEDIESPWDSESISENFPQKYVDPLAGAADGKEKNIGNEQAEDVFYIPSCMSGSRNFKMAKLEDTRNVGMPVAHMESPERYLHLKPTIEMKDSVPNKAGGMKDVQTSKAAEHDLEVASEEEQEREGSENNQPQVEEERKKHRNNEMEVSANIHDGATDDAEDDDDDDGLIQKRKSGETDHQQFPRKENKEYASSGPALQMKEVKSTEKEKRTSKESVNSPVFGKASLLTGGLLQVDDDSSLSEIDEDEGRPTKKTSNEKNKVKNQIQSMDDVDDLTQSSETASEDCELPHSSYKNFMLLIEQLGMECKDSVSLLKIQDAALSCERLLELKKNHCELLTVKIKKMEDKVNVLQRELSETKEIKSQLEHQKVEWERELCSLRFSLNQEEEKRRNADTLYEKIREQLRRKEEQYRKEVEVKQQLELSLQTLEMELRTVKSNLNQVVQERNDAQRQLSREQNARMLQDGILTNHLSKQKEIEMAQKKMNSENSHSHEEEKDLSHKNSMLQEEIAMLRLEIDTIKNQNQEKEKKCFEDLKIVKEKNEDLQKTIKQNEETLTQTISQYNGRLSVLTAENAMLNSKLENEKQSKERLEAEVESYHSRLAAAIHDRDQSETSKRELELAFQRARDECSRLQDKMNFDVSNLKDNNEILSQQLFKTESKLNSLEIEFHHTRDALREKTLGLERVQKDLSQTQCQMKEMEQKYQNEQVKVNKYIGKQESVEERLSQLQSENMLLRQQLDDAHNKADNKEKTVINIQDQFHAIVQKLQAESEKQSLLLEERNKELISECNHLKERQYQYENEKAEREVVVRQLQQELADTLKKQSMSEASLEVTSRYRINLEDETQDLKKKLGQIRNQLQEAQDRHTEAVRCAEKMQDHKQKLEKDNAKLKVTVKKQMDKIEELQKNLLNANLSEDEKEQLKKLMELKQSLECNLDQEMKKNVELEREITGFKNLLKMTRKKLNEYENGEFSFHGDLKTSQFEMDIQINKLKHKIDDLTAELETAGSKCLHLDTKNQILQEELLSMKTVQKKCEKLQKNKKKLEQEVINLRSHIERNMVELGQVKQYKQEIEERARQEIAEKLKEVNLFLQAQAASQENLEQFRENNFASMKSQMELRIKDLESELSKIKTSQEDFNKTELEKYKQLYLEELKVRKSLSSKLTKTNERLAEVNTKLLVEKQQSRSLFTTLTTRPVMEPPCVGNLNNSLDLNRKLIPRENLVISTSNPRASNNSMENYLSKMQQELEKNITRELKEAAAELESGSIASPLGSTDESNLNQDLVWKASREYVQVLKKNYMI

SEQ ID NO: 10 - DNA, 6816 bp, homo sapiens
acttgtgtctagcgggatcgcttgcttggtaacccggagggagagattggaaaccgcggagtttcctttgggaggctgcggccagccggggctgacttgttatgttgggctccggaggccgttaagagccgagagagacatgaggtgtctctgaagcccggtcgcctgggccatgaagaagatttttagtaagaagggcgagtcgcccttgggctccttcgcgcggcggcagaggagcagcgcgggaggcgggggcgagccgggggagggcgcctactcgcagcccggctaccacgtccgagaccgagatctcggcaagatccacaaagctgccagcgcgggtaatgtggcgaaagtgcagcagatccttttgctcaggaagaatggcttgaacgatagagacaagatgaacaggacggctctacatttggcctgtgccaatggtcatccagaagtagtaactctcctggtggacagaaaatgccagctcaatgtctgtgacaacgaaaacaggacagctctgatgaaggctgtacaatgccaggaagagaaatgtgcaactattctgctagaacatggtgctgatccaaatcttgcggatgtccatggcaacactgctcttcactatgctgtctataatgaggacatatcagtagcaacaaagctgcttttgtatgatgcaaatattgaagcaaaaaacaaggatgacctcacaccacttttacttgcagtaagtggaaaaaagcagcaaatggtggaatttttaataaagaaaaaagcaaatgtaaatgcagtagataagttggaaagcagtcaccaactaatttcagaatataaagaagaaaggatacctaaacattcttctcaaaatagtaattcagtggatgaaagctctgaagactccttaagcaggctttctggcaaaccgggtgttgatgattcatggcctacctcagatgacgaagacctcaattttgatactaagaatgtcccaaaaccaagcttagcaaagctaatgactgcttctcagcaatccaggaaaaatttagaagcaacatatggcactgtgagaacaggaaatagaactttgtttgaggatagagattccgatagtcaagatgaagttgtggttgaaagccttcctacaacatcaatcaaagtccagtgcttttctcatcctacctatcaatcacctgaccttcttccaaaaccttcccacaagtcgttagcaaaccctggtcttatgaaggaagaaccaacaaagccaggcattgcaaaaaaagaaaatggtattgatattattgaaagtgctccactagagcaaacaaataatgacaatttgacttatgttgatgaagtgcacaaaaataatagaagtgatatgatgtccgcattaggattaggacaagaggaagatatagaatcaccttgggattctgagagtatctctgagaattttccacagaagtatgttgatcctttagctggggctgcagacggaaaagaaaaaaatataggaaatgaacaagcagaagatgtgttttatataccttcttgcatgagtggatcaagaaactttaagatggctaaactagaggatacaagaaatgtaggcatgccagtagcccacatggagtctcctgagagatatcttcacttgaagcctaccattgaaatgaaagattctgttccaaataaagcaggaggaatgaaggatgtacaaacatccaaagcagctgaacatgacttagaagtagcatcagaagaagagcaagaaagggaagggagtgaaaataaccagccacaggttgaagaagaaaggaaaaaacacagaaataatgaaatggaagtatcagcaaacatacatgatggtgctactgatgatgctgaagatgatgatgatgatgatggattaattcaaaaaagaaagagtggagaaactgatcatcagcaatttcccaggaaggaaaataaagagtatgctagtagtggtcctgccttgcaaatgaaggaagtaaagagcactgaaaaagaaaaacggacctcgaaagaatctgtgaattcaccagtgtttgggaaggccagtttactaactggtggcctgctacaagtggatgatgacagcagtttaagtgaaatagatgaggatgaaggaaggcctactaagaaaacatctaatgaaaagaacaaggtcaaaaaccaaatacagtctatggatgatgttgatgacttaactcagtcatctgaaacagcctcagaggattgtgagctaccccactctagttacaagaattttatgttgctcattgaacaacttggaatggagtgtaaagattctgttagcctattgaaaatccaggatgcagctctttcatgtgaaagattattagaacttaaaaaaaatcactgtgaactacttacagtaaaaattaaaaaaatggaagacaaggttaatgtactacaaagggagctatctgaaacaaaagaaataaaatcacagttagagcatcaaaaagttgaatgggaacgagaactgtgctctttgagatttagcttaaaccaagaagaagagaagagaagaaatgctgatacgttgtatgaaaaaattagggaacagttaagaagaaaagaagagcaatataggaaagaagttgaagtgaaacaacagcttgaactgagtctccaaacactggagatggaattgaggactgtaaaaagtaatttgaatcaggtcgttcaagagcgaaatgacgctcagaggcaactttctcgagaacagaatgccagaatgttacaagatggaattctgaccaatcacctttccaaacaaaaggagattgaaatggctcaaaagaaaatgaattctgagaattctcatagtcatgaagaagaaaaagacctatcgcataaaaatagcatgttgcaggaagaaattgctatgctaagactagaaatagacacaataaaaaatcaaaaccaggaaaaagaaaagaaatgttttgaggaccttaaaattgtaaaagaaaagaatgaagaccttcagaagactataaaacagaatgaggaaacattaacacaaacaatatcccagtataatggacggcttagtgttctgacagctgagaatgcaatgctaaattctaaactggagaatgaaaagcaaagcaaggaaagactggaagcagaagttgaatcataccattctagattggctgctgctatacatgatcgtgatcaaagtgagacatcaaaaagagaactagaacttgctttccagagagcaagagatgaatgttctcgtttacaggacaaaatgaattttgatgtgtctaacctaaaagataacaatgagattctttctcaacaactatttaaaactgaaagtaaactcaatagcctagaaattgagttccatcacacgagagatgccctcagagaaaagactttgggtttagaacgggtacaaaaggacctaagccaaacacagtgtcaaatgaaggaaatggaacaaaagtatcaaaatgaacaagttaaagtgaataaatacattggaaagcaggagtctgtagaggagagattgtctcaactacaaagtgagaatatgttgcttcgacaacaactggatgatgcccacaacaaggctgacaataaagagaagacagtgattaatatccaagaccagtttcatgctattgtgcaaaaacttcaagctgagagtgaaaagcaaagtcttctgctagaagaaagaaataaggagttaatcagtgaatgtaatcacttaaaagaaagacagtatcaatatgaaaatgaaaaggcagaaagagaagttgttgtgagacaacttcaacaagaactagctgataccctaaaaaaacaatctatgtcagaggcttcactggaggttacgtcacgttatcgtattaatttagaagatgagacacaggatttaaagaagaaattaggtcaaatcagaaatcaattgcaagaagcacaggatcgacatacagaagctgtcagatgtgctgagaagatgcaagatcacaagcaaaagcttgaaaaagataatgccaagttaaaagttacagtcaaaaagcaaatggacaaaattgaggagcttcagaaaaacctgttaaatgcaaatttgtctgaagatgaaaaggaacaattaaagaaacttatggaattaaaacagtcactggaatgtaatttggatcaagaaatgaagaaaaatgttgaattagaaagagagataactggatttaagaacctcttaaaaatgacaagaaagaagttaaatgaatatgaaaatggagaatttagtttccatggagatttaaaaactagtcaatttgaaatggatattcagattaataagctaaaacataagattgatgatcttacagcagaactggagactgcaggttcaaaatgtctacatctggatacaaagaaccaaattcttcaagaggagttgttatctatgaaaacagtacaaaagaaatgtgaaaaactacagaagaataaaaagaagttggaacaagaagtgatcaacctgagaagtcatatagaaaggaatatggtagaacttggtcaagtcaaacagtataaacaggagattgaagaaagagcaagacaggaaatagcagaaaaattaaaagaagtcaatctatttttacaggcacaagcagcatctcaagaaaacttagagcagtttagagagaataattttgcttcaatgaaaagtcagatggaactcagaattaaagatctggaatctgaactctccaaaataaaaacttctcaagaagactttaataaaaccgaactggaaaaatataagcaactctatctagaagaattaaaagttagaaaatctttgtcaagtaaactaaccaaaactaatgagaggctagcagaggtcaacaccaaacttcttgtggaaaaacagcagagcagatctttgttcaccactctcactaccaggccagtcatggagccaccttgtgtgggaaatcttaataatagtttagatctcaacagaaaacttattccaagagaaaacttagtgatctctacctcaaatccacgggcttcaaataatagcatggagaactacttgagcaagatgcagcaggagttggaaaaaaatataactagagaactcaaagaagctgctgctgaattggaatctggatcaatagcttcccctctagggtctactgatgagtcaaatctaaatcaagatctagtttggaaagcatcaagagaatatgtacaggttttaaagaaaaattatatgatctgaaagataagattttattactgggctgtttatgtgacattttgtttctcattaaatattaatatatgacatgtaaaaatattaaaggaacgtatttttgtatcatatatgtcagatcaaaatttatatagtattttaaacaatgttacttggcttctttaacttttaagtggattttgccaatgaaaaccaggaatattgagttttacatactcaaactgcccaaatgtgagctgtttaaacagccaaaccactaagttgtcattaatactttagtgcagttaattgatggcttatttgtattttgtaatttttatcactatatgtagtggtagatgtctaattggacttagacatcattttgttgtgcaggaactgtggtcattgttcatgtttggatgtattacaattgttacagtgccatcaaatttacataattttaattttaagcactgatttatatagctgttctcagggagaaacaagatttgcctgattcaccttttctgtttgtttatttatttttaattaaccttttggaaatagccttcagtttagagaaaatttcgaagttttagtacaaagactttctcctccgaacaatttgagagtaaattgctgacattatgtcatcacccctgattactttagtgggcattcctacaagtcaggacattctacataactacagtgcaaaccttgcaatcagaaagtcaacattaatacattctagtttgttacttctctgtaatgtcctttatagcaaaaggatcccattctaatccatggcttgtatttagttgttgtatctgttagatctttcagtctggaacagttcctcagtctttccttgaccttcatgaccttgaatattttgaagagtaaagtctggtaatataagaatgtccctcaattgggtttcatggtgattagatttagattatatgattttttggcaggagtatcacagaagtgacccagtgatcttctcattgcatcttacctgtgaccctacagttttgatttgccctgttactggtaatgttagcattgatcactaacagtggtatctgctgaatttctcctctgtaatgttattttttcaccttgtggttaattagtattttacagggaggtaatttgagactatgtacatatctctactccccatcgaaccctcaatttattcatttgcttgtatcaaataaaccatggattcatggtttattttattcagtgagttataattcattaatattgttattaaaaatttgtttttcttttaagaaatgttatattttgacaacttcagacttacagaaagattataagaggtagtacaaataatttttggatattattccccaaatgttaacattttactgcatttactttatcctttctcccccttctcctcctgtctttctagatgaatatgaatataggtacttaatacagattttttttctaaactgttaaaaaaaaaaaaaaaaaaaaaaaaaaaaaaaaaaaaa

(19) United States
(12) Patent Application Publication
(10) Pub. No.: US 2015/0232932 A1
(43) Pub. Date: Aug. 20, 2015

(54) ANKRD26 AS A MARKER FOR DIAGNOSIS OF THROMBOCYTOPENIAS

(71) Applicant: INSTITUT GUSTAVE ROUSSY, Ville juif (FR)

(72) Inventors: Dominique Bluteau, Paris (FR); Remi Favier, Sceaux (FR); Hana Raslova, Le Coudry Montceaux (FR); William Vainchenker, Paris (FR)

(73) Assignee: INSTITUT GUSTAVE ROUSSY, Ville juif (FR)

(21) Appl. No.: 14/428,649

(22) PCT Filed: Sep. 17, 2013
(86) PCT No.: PCT/EP2013/002799

§ 371 Date: Mar. 17, 2015

(30) Foreign Application Priority Data
Sep. 17, 2012 (EP) 12006529.7

(51) Int. Cl. C12Q 1/68; G01N 33/68

(52) U.S. Cl. CPC C12Q 1/6883; G01N 33/6893; C12Q 2600/158; G01N 2333/4703; G01N 2800/226; G01N 2800/222; G01N 2333/4712

(57) ABSTRACT

An in vitro method for diagnosing thrombocytopenia in a subject, the method includes the step of assessing the ANKRD26 platelet expression in a biological sample from the subject, wherein a ANKRD26 platelet expression is indicative of a thrombocytopenia. Also described is a kit for diagnosing a thrombocytopenia in a subject, which includes at least one ANKRD26 nucleic acid probe or oligonucleotide or antibody for assessing the ANKRD26 platelet expression in a biological sample from the subject.

This international patent application claims the priority of European patent application EP 12006529.7 filed on Sep. 17, 2012, which is herein incorporated by reference.

### FIELD OF THE INVENTION

The present invention relates to ANKRD26 as a biomarker for diagnosing thrombocytopenias such as THC2 and FPD/AML.

### BACKGROUND OF THE INVENTION

Inherited thrombocytopenias constitute a heterogeneous group characterized by a reduced number of blood platelets and a bleeding tendency ranging from very mild to life threatening. The genetic defects associated with the pathology have been identified for some of these pathologies, whereas they are still unknown for the others. These defects generally consist in a mutation in a gene implied in the process of megakaryopoiesis and platelet production.

A significant proportion of patient affected by an inherited thrombocytopenia remain without a definite diagnosis. Making a definitive diagnosis for these patients remains important because the different forms of these pathologies imply different prognosis, therapy and management. Indeed, for example, some inherited thrombocytopenias, such as familial platelet disorder with propensity for myeloid malignancy (FPD/AML) or thrombocytopenia 2 (THC2), significantly increase the risk of leukemia.

Thrombocytopenia 2 (THC2; OMIM 188000) is one of the rarest form of familial autosomal-dominant thrombocytopenias, for which mutations of MASTL and ACBD5 genes have been identified as causative defects. THC2 is characterized by a moderate thrombocytopenia, reduction of platetlet α-granules, normal platelet aggregation and normal mean platelet volume, and seems to be derived from dysmegakaryopoiesis. Recently, mutations in a short stretch of the 5′-untranslated region (5′-UTR) of the ANKRD26 gene have been identified and associated with this particular form of thrombocytopenia (Pippucci T. et al, The American Journal of Human Genetics 88, 115-120, Jan. 7, 2011; Norris P; et al, Blood, 16 Jun. 2011, Volume 117, Number 24): these studies indicate that the ANKRD26 mutations cause thrombocytopenia and might enhance ANKRD26 expression.

Familial platelet disorder with propensity to acute myeloid leukaemia (FPD/AML; OMIM 601399) is also an autosomal-dominant disorder in which affected members have a clinical history of bleeding tendency and mild to moderate thrombocytopenia with normal platelet size and morphology, and/or abnormal platelet aggregation in response to arachidonic acid (HO et al., Blood, vol. 87, p: 5218-5224, 1996). FPD/AML has been associated with RUNX1 (Song et al., Nat Genet, vol. 23, p: 166-175, 1999; Michaud et al., Blood, vol. 99, p: 1364-1372, 2002). FPD/AML diagnosis is currently based on sequencing the complete RUNX1 gene sequence and new biomarkers of the pathology recently appeared such as MYH10 (Antony-Debré I et al, Blood. 2012 Jun. 7).

FPD/AML and THC2 are both familial autosomal-dominant thrombocytopenias characterized by the same clinical symptoms and a predisposition to leukemias. It would be of great interest to dispose of methods or biomarkers permitting a simple diagnosis, and more particularly a differential diagnosis, of these both pathologies.

### SUMMARY OF THE INVENTION

The present inventors interestingly discovered that the ANKRD26 protein is present in platelets of THC2 and FPD/AML patients, whereas it is absent in platelets of healthy subjects. They also showed that MYH10 is present in platelets of FPD/AML patients, whereas it is absent in platelets of THC2 patients and healthy subjects.

Thus, the present invention relates to an in vitro method for diagnosing thrombocytopenia in a subject, said method comprising the step of (i) assessing the ANKRD26 platelet expression in a biological sample from said subject, and wherein an ANKRD26 platelet expression is indicative of a thrombocytopenia.

Particularly, the invention relates to an in vitro method as described above, wherein said method is used for distinguishing THC2 and FPD/AML and said method further comprises step of (ii) assessing the MYH10 platelet expression in a biological sample from said subject, and wherein:
a. an ANKRD26 platelet expression and no MYH10 platelet expression is indicative of a THC2, and
b. an ANKRD26 platelet expression and a MYH10 platelet expression is indicative of a FPD/AML.

The invention also provides a kit for diagnosing a thrombocytopenia in a subject according to the methods of the invention, which comprises (i) at least one ANKRD26 nucleic acid probe or oligonucleotide or antibody for determining the ANKRD26 platelet expression in a biological sample from said subject.

Particularly, the invention provides a kit as described above, wherein said kit further comprises (ii) at least one MYH10 nucleic acid probe or oligonucleotide or antibody for further assessing the MYH10 platelet expression in a biological sample from said subject.

### BRIEF DESCRIPTION OF DRAWINGS

The FIG. 1 shows ANKRD26 expression level during megakaryocyte differentiation. CD34+ cells (D0) were obtained from cytapheresis and cultures in presence of thrombopoietin (TPO) alone to induce megakaryocyte differentiation. The cells were collected at day 1, 4, 7 and 10 of culture in the experiment represented in the left panel, at day 6, 9 and 12 of culture in the experiment represented in the middle panel and megakaryocytes were sorted according to CD41 and CD42 expression at day 6 of culture in the experiment represented in the right panel. CD41+CD42− represent the immature megakaryocyte CD41+CD42+ represent more mature megakaryocytes. The RNA expression level of ANKRD26 was measured by Q-RT-PCR and its level is represented relative to PPIA.

The FIG. 2 shows ANKRD26 expression in megakaryocytes of THC2 patients and platelets of THC2 and FPD/AML patients.

FIG. 2A: CD34⁺ cells were isolated from THC2 patient's (P1, P2, P3) or healthy individual's (C1, C2) peripheral blood and cultured in presence of thrombopoietin. CD41⁺CD42⁺ megakaryocytes were sorted at day 10 of culture and ANKRD26 expression level was measured by Q-RT-PCR, normalized to PPIA and expressed relatively to C1. P2 and P2b represent two different RNA extractions.

FIG. 2B: THC2 patient's (ANKRD26 patients, P1-P6), FPD/AML patient's (RUNX1 mutations, P1, P2) and healthy individual's (C1-C5) platelets were obtained from platelets-rich plasma. ANKRD26 and MYH10 expression level was measured by Q-RT-PCR, normalized to PPIA and expressed relatively to C1.

The FIG. 3 illustrates a diagram of the double screening of MYH10 and ANKRD26 expression to distinct between the pathologies presenting similar clinical phenotype.

### DETAILED DESCRIPTION OF THE INVENTION

Methods of the Invention

A first object of the invention relates to an in vitro method for diagnosing thrombocytopenia in a subject, said method comprising the step of (i) assessing the ANKRD26 platelet expression in a biological sample from said subject, and wherein an ANKRD26 platelet expression is indicative of a thrombocytopenia.

The term “ANKRD26” has its general meaning in the art and refers to a gene encoding in humans the Ankyrin repeat domain-containing protein 26 or ANKRD26 protein (also called THC2 gene or bA145E8.1). The term may include naturally occurring ANKRD26 and variants and modified forms thereof. The ANKRD26 can be from any source, but typically is a mammalian (e.g., human and non-human primate) ANKRD26, preferably a human ANKRD26. An exemplary native ANKRD26 amino acid sequence is provided in GenPept database under accession number NP_{—}001242982 (SEQ ID NO:1) or NP_{—}055730 (SEQ ID NO:9) and an exemplary native nucleotide sequence encoding for ANKRD26 is provided in GenBank database under accession number NM_{—}001256053 (SEQ ID NO:2) or NM_{—}014915 (SEQ ID NO:10).

Thrombocytopenias are well known from the skilled person and include familial thrombocytopenia 2 called THC2 and familial platelet disorder with propensity to acute myeloid leukemia called FPD/AML. These both forms of thrombocytopenia are associated with a predisposition to leukemia such as acute myeloid leukemia (AML) and chronic myelomonocytic leukemia (CMML).

In a particular embodiment, thrombocytopenia is THC2. In another embodiment, thrombocytopenia is FPD/AML.

As used herein, the term “subject” denotes a mammal, such as a rodent, a feline, a canine, and a primate. Preferably, a subject according to the invention is a human.

As used herein, the term “biological sample” refers to a sample in which platelets ANKRD26 expression level may be assayed. Biological samples comprising platelets are well known from the skilled person and include blood samples and bone marrow samples. Preferably, the biological sample is a blood sample.

In a particular embodiment, the in vitro method of the invention is used for distinguishing THC2 and FPD/AML. It further comprises a step of (ii) assessing the MYH10 platelet expression in said biological sample (or in a biological sample obtained from said subject).

In this particular embodiment, the combination of (a) a ANKRD26 platelet expression and (b) no MYH10 platelet expression is indicative of THC2; the combination of (a′) a ANKRD26 platelet expression and (b′) a MYH10 platelet expression is indicative of FPD/AML.

The term “MYH10” has its general meaning in the art and refers to the gene encoding the myosin non-muscle heavy chain 10 protein or MYH10 protein (Gene ID: 4628, also known as NMMHCB, MGC134913 or MGC134914). The term may include naturally occurring MYH10 and variants and modified forms thereof. The MYH10 can be from any source, but typically is a mammalian (e.g., human and non-human primate) MYH10, preferably a human MYH10. An exemplary native MYH10 amino acid sequence is provided in GenPept database under accession number NP_{—}005955 (SEQ ID NO: 3) and an exemplary native nucleotide sequence encoding for MYH10 is provided in GenBank database under accession number NM_{—}005964 (SEQ ID NO: 4).

The platelets expression level of a gene or a protein in a biological sample of the invention may be assessed by purifying platelets from said biological sample.

Methods for purifying platelets from biological samples such as blood samples are well known from the skilled person and are disclosed, as an example, in NIUL et al. (Comparative Haematology International, vol. 2(2), p: 106-110, 1992), GNATENKO et al. (Methods Mol. Biol., vol. 496, p: 245-72, 2009), in SCHEDEL & ROLF (Methods Mol. Biol., vol. 496, p: 273-83, 2009), or in CHOI et al. (Lab. Chip., “Hydrophoretic high-throughput selection of platelets in physiological shear-stress range”, 2010).

The subject may be healthy, but the method of the invention is particularly useful for testing a subject thought to develop or to be predisposed to developing thrombocytopenia chosen among the group comprising THC2 and FPD/AML. In that case, the method of the invention enables to confirm that said subject develops or is predisposed for developing THC2 or FPD/AML.

In a particular embodiment, the method of the invention may be used for prognosing a predisposition to AML or CMML in a subject.

In a preferred embodiment, the method of the invention further comprises the step of comparing said ANKRD26 and/or MYH10 platelet expression in said biological sample with a control.

As used herein said “control” refers to the platelet ANKRD26 and/or MYH10 expression level in a control sample corresponding to the ANKRD26 and/or MYH10 expression level in platelets of a biological sample from a healthy subject. Said control is preferably the average expression levels of ANKRD26 and/or MYH10 in several control biological samples. As determined by the inventors, there is no expression of ANKRD26 and/or MYH10 in the platelets of healthy subjects.

Thus, as used herein, an “ANKRD26 and/or MYH10 platelet expression” means a significant ANKRD26 and/or MYH10 platelet expression (or expression level) compared to said control expression (or expression level), by any method for assessing a platelet ANKRD26 and/or MYH10 expression (or expression level).

A significant ANKRD26 and/or MYH10 platelet expression (or expression level) compared to said control level refers to an expression at least two, particularly at least three, more particularly at least five, preferably at least ten times higher than the ANKRD26 and/or MYH10 platelet control expression (or expression level).

Methods for determining the ANKRD26 and/or MYH10 expression levels in platelets are well known for the man skilled in the art.

In one particular embodiment, the level of expression of the ANKRD26 and/or MYH10 gene(s) is assessed by determining the level of expression of the ANKRD26 protein (i.e. SEQ ID N° 1 or SEQ ID N° 9) and/or the MYH10 protein (i.e. SEQ ID N° 3).

Methods for determining the level of expression of a protein are well known in the art. Such methods comprise contacting a biological sample with a binding partner capable of selectively interacting with the protein of interest present in the sample. The binding partner is generally an antibody.

Thus, such analysis can be assessed an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the ANKRD26 and/or MYH10 protein respectively. Said analysis can be assessed by a variety of techniques well known by one of skill in the art including, but not limited to, enzyme immunoassay (EIA), radioimmunoassay (RIA), Western blot analysis and enzyme linked immunoabsorbant assay (ELISA).

Polyclonal antibodies can be prepared by immunizing a suitable animal, such as mouse, rabbit or goat, with the ANKRD26 protein (SEQ ID NO: 1 or SEQ ID N° 9) and/or MYH10 protein (SEQ ID NO:3) or a fragment thereof (e.g., at least 10 or 15 amino acids). The antibody titer in the immunized animal can be monitored over time by standard techniques, such as with an ELISA using immobilized polypeptide.

ANKRD26 or MYH10 antibodies are already commercially available from SANTA CRUZ BIOTECHNOLOGY, INC., LIFESPAN BIOSCIENCES, or from SIGMA-ALDRICH.

At an appropriate time after immunization, e.g., when the specific antibody titers are highest, antibody producing cells can be obtained from the animal and used to prepare monoclonal antibodies (mAb) by standard techniques, such as the hybridoma technique originally described by KOHLER and MILSTEIN (Nature, vol. 256, p: 495-497, 1975), the human B cell hybridoma technique (KOZBOR et al., Immunol., vol. 4, p: 72, 1983), the EBV-hybridoma technique (COLE et al., In Monoclonal Antibodies and Cancer Therapy, Alan R. Liss, Inc., p: 77-96, 1985) or trioma techniques. The technology for producing hybridomas is well known (see generally Current Protocols in Immunology, COLIGAN et al. ed., John Wiley & Sons, New York, 1994). Hybridoma cells producing the desired monoclonal antibody are detected by screening the hybridoma culture supernatants for antibodies that bind the polypeptide of interest, e.g., using a standard ELISA.

In another particular embodiment of the invention, the level of expression of the ANKRD26 and/or MYH10 gene is assessed by determining the level of expression of its mRNA transcript (i.e., SEQ ID N° 2 or SEQ ID N° 10 for ANKRD26 and SEQ ID NO:4 for MYH10) or mRNA precursors, such as nascent RNA, of said gene.

Methods for determining the quantity of mRNA are well known in the art.

Such analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TAQMAN), and probes arrays such as GENECHIP™ DNA Arrays (AFFYMETRIX).

Advantageously, the analysis of the expression level of mRNA transcribed from the ANKRD26 and/or MYH10 gene involves the process of nucleic acid amplification, e.g., by RT-PCR (the experimental embodiment set forth in U.S. Pat. No. 4,683,202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 87, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

Preferably, a pair of primers permitting to amplify ANKRD26 has the sequences SEQ ID N° 5 and SEQ ID N° 6 and a pair of primers permitting to amplify MYH10 has the sequences SEQ ID N° 7 and SEQ ID N° 8.

In a preferred embodiment, the invention relates to an in vitro method for diagnosing THC2 in a subject, said method comprising the step of (i) assessing the ANKRD26 platelet expression in a biological sample from said subject, and wherein a ANKRD26 platelet expression is indicative of THC2.

In a more preferred embodiment, said method further comprises a step of (ii) assessing the MYH10 platelet expression in said biological sample (or in a biological sample obtained from said subject), wherein no MYH10 platelet expression is indicative of THC2.

Kits of the Invention

A second object of the invention relates to a kit for diagnosing a thrombocytopenia in a subject according to the methods of the invention, which comprises (i) at least one mean for assessing the ANKRD26 platelet expression in a biological sample from said subject.

In a particular embodiment, said kit comprises an ANKRD26 nucleic acid probe or oligonucleotide or antibody for assessing the ANKRD26 platelet expression in a biological sample from said subject.

Preferably, the oligonucleotide is at least one PCR primer pair, most preferably SEQ ID NO: 5 and SEQ ID NO: 6.

In one embodiment, the kit of the invention further comprises (ii) at least one mean for assessing the MYH10 platelet expression in a biological sample from said subject.

In a particular embodiment, said kit comprises a MYH10 nucleic acid probe or oligonucleotide or antibody for further assessing the MYH10 platelet expression in a biological sample from said subject.

Preferably, the oligonucleotide is at least one PCR primer pair, most preferably SEQ ID NO: 7 and SEQ ID NO: 8.

The kit of the invention can further comprise at least one mean for purifying platelets from a biological sample, preferably from a blood sample.

The kit of the invention may be used for diagnosing a thrombocytopenia in a subject according to the methods of the invention, wherein said thrombocytopenia is preferably THC2 or FPD/AML. The invention thus relates to the use of a kit of the invention for diagnosing a thrombocytopenia, preferably THC2 or FPD/AML.

In a particular embodiment, the kit of the invention may be used for testing a subject thought to develop or to be predisposed to developing a thrombocytopenia, wherein said thrombocytopenia is preferably FPD/AML or THC2. The invention thus relates to the use of a kit of the invention for testing a subject thought to develop or to be predisposed to developing a thrombocytopenia, preferably THC2 or FPD/AML.

In another particular embodiment, the kit of the invention may be used for prognosing a predisposition to AML and/or CMML in a subject. The invention thus relates to the use of a kit of the invention for prognosing a predisposition to AML and/or CMML in a subject.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

The present kits can also include one or more reagents, buffers, hybridization media, nucleic acids, primers, nucleotides, probes, molecular weight markers, enzymes, solid supports, databases, computer programs for calculating dispensation orders and/or disposable lab equipment, such as multi-well plates, in order to readily facilitate implementation of the present methods. Enzymes that can be included in the present kits include nucleotide polymerases and the like. Solid supports can include beads and the like whereas molecular weight markers can include conjugable markers, for example biotin and streptavidin or the like.

In one embodiment, the kit is made up of instructions for carrying out the method described herein. The instructions can be provided in any intelligible form through a tangible medium, such as printed on paper, computer readable media, or the like.

In the following, the invention is described in more detail with reference to amino acid sequences, nucleic acid sequences and the examples. Yet, no limitation of the invention is intended by the details of the examples. Rather, the invention pertains to any embodiment which comprises details which are not explicitly mentioned in the examples herein, but which the skilled person finds without undue effort.

### EXAMPLES

### Material and Methods

Patients.

The study was approved by the Local Research Ethics Committee from AP-HP (Assistance Publique des Hôpitaux de Paris) and INSERM. Blood samples from patients and healthy subjects were collected with informed consent in accordance with the Declaration of Helsinki.

In Vitro Megakaryocyte Culture Derived from Human CD34+ Cells in Liquid Serum-Free Medium.

CD34+ cells and platelets were obtained in agreement with our Institute Ethic Committee (AP-HP) from the peripheral blood of healthy patients undergoing hip surgery and from leukapheresis samples after mobilization. CD34+ cells were isolated by an immunomagnetic cell sorting technique (AUTOMACS; MILTENYI BIOTEC, Bergisch Gladbach, Germany) and cultured in serum-free medium in the presence of recombinant human thrombopoietin (TPO, 10 ng/mL; KIRIN BREWERY, Tokyo, Japan) for inducing megakaryocyte differentiation. Megakaryocytes were sorted according to CD41 and CD42 expression using a FACSDIVA or Influx flow cytometer (BECTON DICKINSON, Mountain View, Calif.).

Platelets

The platelet-rich plasma was prepared by centrifugation at 900 rpm for 10 min. Platelets were pelleted by centrifugation at 3,000 rpm for 10 min.

Quantitative Real-Time PCR (RT-PCR).

RNA was isolated using micro RNeasy extraction kit (QUIAGEN), reverse-transcribed by Vilo Superscript kit (INVITROGEN) and Q-RT-PCR were carried out with a PRISM® 7700 sequence detection system using SYBR® green (APPLIED BIOSYSTEMS). The expression levels of all genes studied were expressed relatively to housekeeping genes PPIA.

Western Blot.

The proteins are extracted from megakaryocytes at day 6, 9 and 12 of culture in presence of TPO, from platelets of healthy controls and THC2 and FPD/AML patients. The western blot is performed as previously described (Antony-Debré et al, Blood, 2012) using antibodies directed against ANKRD26 (SANTA CRUZ BIOTECHNOLOGY), MYH10 (CELL SIGNALING), and HSC70 (SIGMA). The protein expression level of MYH10 is normalized to HSC70. Super Signal West Femto Maximum Sensitivity Substrate (Thermo Scientific) is used for MYH10 detection and Immobilon Western Chemiluminescent HRP Substrate (Millipore Corporation) for HSC70 detection.

Statistics.

Data are presented as means (±SD). Statistical significance was determined by Student's t test. A P value <0.05 was considered as statistically significant.

### Results

ANKRD26 Expression During Normal Megakaryocyte Differentiation.

Inventors demonstrated that in hematopoietic progenitors (CD34+ cells corresponding to D0), a high level of ANRD26 was detected.

After induction of megakaryopoiesis by thrombopoietin (FIG. 1), the decrease in ANKRD26 expression was detected by Q-RT-PCR. At the end of megakaryocyte culture (D10-D12), the ANKRD26 was quasi absent.

The ANKRD26 was also measured within two different megakaryocyte populations (CD41+CD42− corresponding to immature megakaryocyte and CD41+CD42+ corresponding to more mature megakaryocyte) at the same day of culture (D6). As shown in FIG. 1 (right panel), the level of ANKRD26 was decreased in more mature megakaryocyte compared to the immature megakaryocyte.

These three independent experiments represented on the FIG. 1 (left, middle and right panel) confirmed the downregulation of ANKRD26 during normal megakaryocyte differentiation.

ANKRD26 Persist in Megakaryocytes and Platelets of THC2 Patients.

Inventors further tested the hypothesis that ANKRD26 should persist in patient megakaryocytes and platelets of THC2 patients.

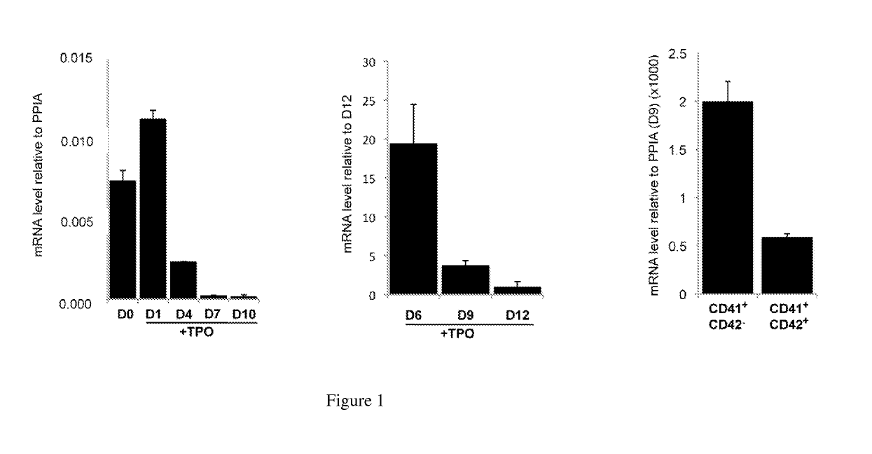

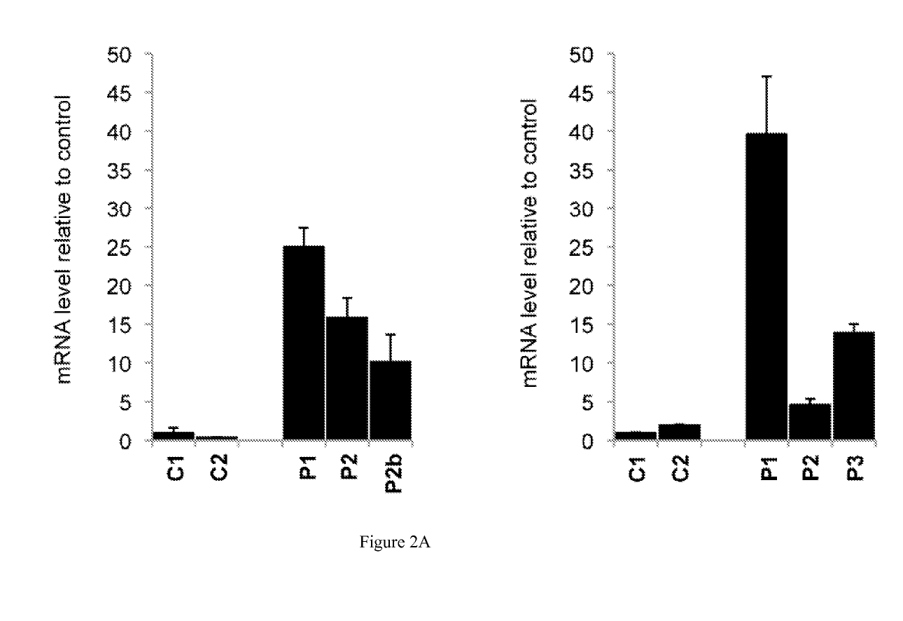

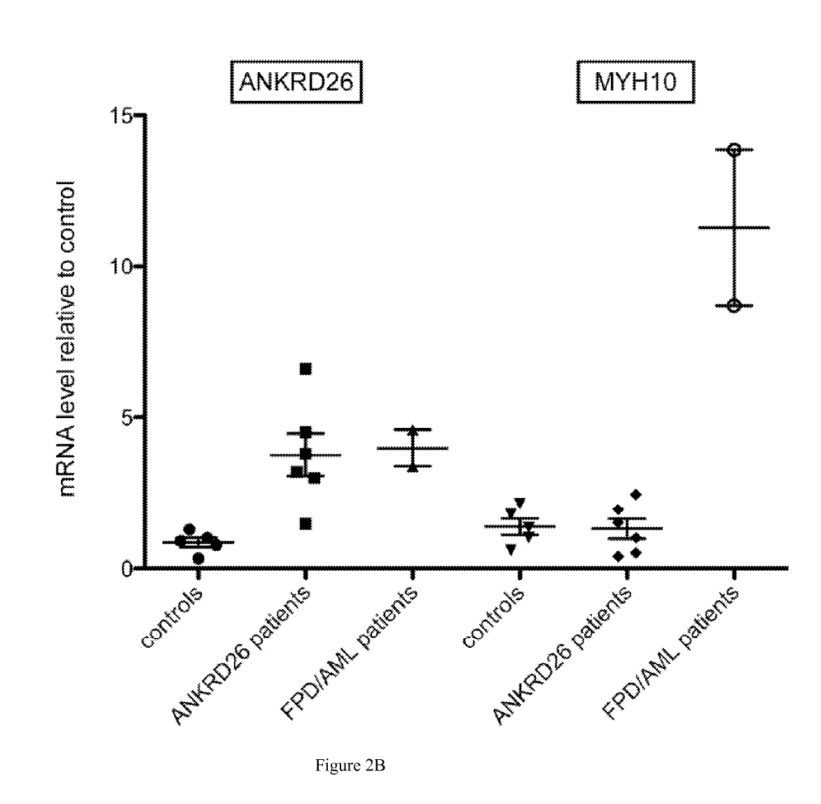

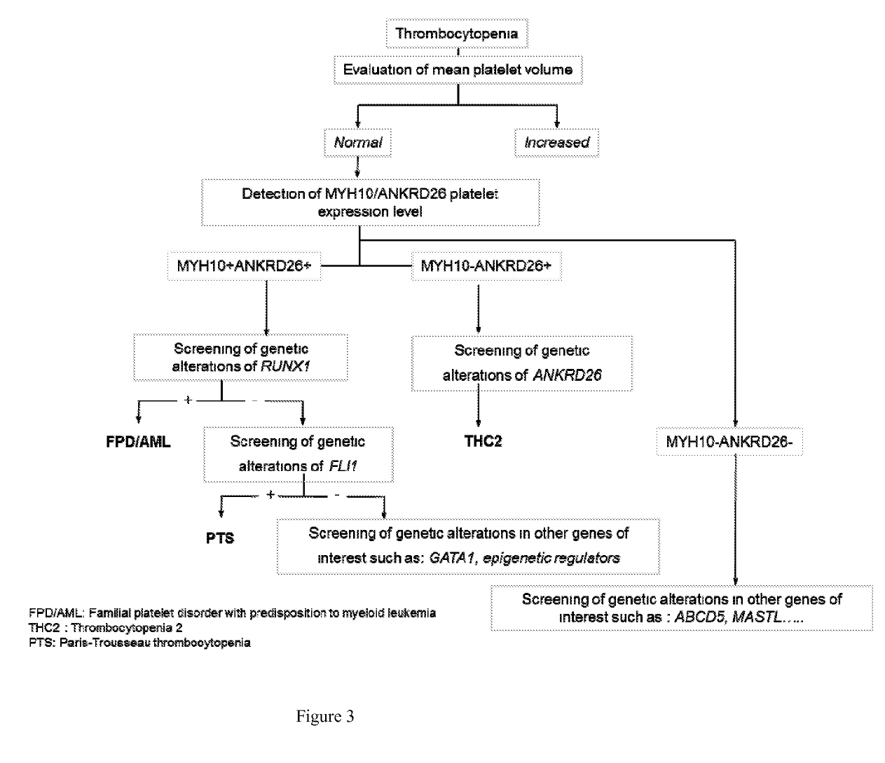

CD34+ hematopoietic progenitors obtained from peripheral blood of three patients (P1 with c.-119C>A mutation, P2 and P3 with c-127A>T mutation) were grown in the presence of thrombopoietin to induce the megakaryocyte differentiation. At day 10 of culture, the mature megakaryocytes (CD41+CD42+ cells) were sorted and the level of ANKRD26 was analysed by Q-RT-PCR. As shown in FIG. 2A, the increased ANKRD26 expression was detected in the megakaryocytes of all three patients (P1, P2 and P2b—two different extractions for P2 at left and P1, P2 and P3 at right) compared to control megakaryocyte.

Then, platelets were obtained from platelet-rich plasma of 6 patients from 3 pedigrees (with c.-119C>A, c-127A>T, c.-118C>A mutations) and ANKRD26 level was detected by Q-RT-PCR. As shown in FIG. 2B, a significantly increased level of ANKRD26 was detected in platelets of all patients.

ANKRD26 also Persist in Megakaryocytes and Platelet of FPD/AML Patients.

The inventors further similarly showed that the ANKRD26 level was also increased in platelets of FPD/AML patients (FIG. 2B).

Previously inventors reported that MYH10 is deregulated in FPD/AML megakaryocytes and platelets in the same manner as ANKRD26 in THC2.

In order to precise the expression pattern of these both biomarkers in thrombocytopenias, inventors analysed the MYH10 expression in platelets of THC2 patients and showed that MYH10 was absent from. THC2 platelets (FIG. 2B).

Assessment of the MYH10 and/or ANKRD26 Protein Level.

As described before, CD34+ hematopoietic progenitors obtained from peripheral blood of three patients (P1 with c.-119C>A mutation, P2 and P3 with c-127A>T) are grown in the presence of thrombopoietin to induce the megakaryocyte differentiation. At day 10 of culture, the mature megakaryocytes (CD41+CD42+ cells) are sorted and the level of ANKRD26 and/or MYH10 protein(s) is analysed by western blot or ELISA.

An increased ANKRD26 expression is detected in the megakaryocytes of THC2 and FPD/AML patients. Furthermore, an increased MYH10 expression is detected in megakaryocytes of FPD/AML patients, but no expression of MYH10 is detected in megakaryocytes of THC2 patients.

Then, platelets are obtained from platelet-rich plasma of THC2 and/or FPD/AML patients and ANKRD26 and/or MYH10 level is detected by ELISA and/or Western Blot.

Similarly, an increased ANKRD26 expression is detected in the platelets of THC2 and FPD/AML patients. Furthermore, an increased MYH10 expression is detected in platelets of FPD/AML patients, but no expression of MYH10 is detected in platelets of THC2 patients.

MYH10 and ANKRD26 as Markers for Diagnosis of Thrombocytopenias.

A double screening of MYH10 and ANKRD26 expression should be thus useful to distinct between these two pathologies presenting similar clinical phenotype; in FPD/AML both MYH10 and ANKRD26 will persist in platelets while in THC2 only ANKRD26 will be detected (FIG. 3).

1. An in vitro method for diagnosing thrombocytopenia in a subject, said method comprising the step of (i) assessing the Ankyrin repeat domain-containing protein 26 (ANKRD26) platelet expression in a biological sample from said subject, wherein a ANKRD26 platelet expression is indicative of a thrombocytopenia.

2. The in vitro method of claim 1, wherein said thrombocytopenia is familial thrombocytopenia 2 (THC2) or familial platelet disorder with propensity for myeloid malignancy (FPD/AML).

3. The in vitro method of claim 1, wherein said subject is a human.

4. The in vitro method of claim 1, wherein said biological sample is a blood sample.

5. The in vitro method of claim 1, wherein said method is used for distinguishing THC2 and FDP/AML and said method further comprises step of (ii) assessing the MYH10 platelet expression in a biological sample from said subject, and wherein:
a. an ANKRD26 platelet expression and no MYH10 platelet expression is indicative of a THC2, and
b. an ANKRD26 platelet expression and a MYH10 platelet expression is indicative of a FPD/AML.

6. The in vitro method of claim 1, wherein said method further comprises the step of comparing said ANKRD26 and/or MYH10 platelet expression in said biological sample with a control.

7. The in vitro method of claim 1, wherein said method is for testing a subject thought to develop or to be predisposed to developing FPD/AML or THC2.

8. The in vitro method of claim 7, wherein said method is for testing a subject thought to be predispose Acute Myeloid Leukemia (AML) and/or to Chronic MyeloMonocytic Leukemia (CMML).

9. A kit for diagnosing a thrombocytopenia in a subject, which comprises (i) at least one ANKRD26 nucleic acid probe or oligonucleotide or antibody for assessing the ANKRD26 platelet expression in a biological sample from said subject.

10. The kit of claim 9 wherein said ANKRD26 oligonucleotide is at least the PCR primer pair defined by the sequences SEQ ID NO: 5 and SEQ ID NO: 6.

11. The kit of claim 9, wherein said kit further comprises (ii) at least one MYH10 nucleic acid probe or oligonucleotide or antibody for further assessing the MYH10 platelet expression in a biological sample from said subject.

12. The kit of claim 11 wherein said MYH10 oligonucleotide is at least the PCR primer pair defined by the sequences SEQ ID NO: 7 and SEQ ID NO: 8.

13. The kit of claim 9, wherein said kit further comprises at least one mean for purifying platelets from the biological sample.

14. The kit of claim 13, wherein said mean is for purifying platelets from a blood sample.

15. Method of using a kit as defined in claim 9 for diagnosing a thrombocytopenia, preferably THC2 or FPD/AML.

16. The in vitro method of claim 2, wherein said subject is a human.

17. The in vitro method of claim 2, wherein said biological sample is a blood sample.

18. The in vitro method of claim 2, wherein said method is used for distinguishing THC2 and FDP/AML and said method further comprises step of (ii) assessing the MYH10 platelet expression in a biological sample from said subject, and wherein:
a. an ANKRD26 platelet expression and no MYH10 platelet expression is indicative of a THC2, and
b. an ANKRD26 platelet expression and a MYH10 platelet expression is indicative of a FPD/AML.

19. The kit of claim 10, wherein said kit further comprises (ii) at least one MYH10 nucleic acid probe or oligonucleotide or antibody for further assessing the MYH10 platelet expression in a biological sample from said subject.
